(19) United States
(12) Patent Application Publication
(10) Pub. No.: US 2005/0107629 A1
(43) Pub. Date: May 19, 2005

(54) Method for producing ethyl acrylate

(76) Inventors: Brian Lee Hershberger, Bourbonnais, IL (US); Lucas John Kammerzell, Humble, TX (US); William Eugene VanArsdale, Seabrook, TX (US)

Correspondence Address: ROHM AND HAAS COMPANY;PATENT DEPARTMENT, 100 INDEPENDENCE MALL WEST, PHILADELPHIA, PA, 19106-2399, US

(21) Appl. No.: 10/968,612

(22) Filed: Oct. 19, 2004

Related U.S. Application Data
(60) Provisional application No. 60/520,806, filed on Nov. 17, 2003.

(51) Int. Cl. C07C 69/52

(52) U.S. Cl. 560/205

(57) ABSTRACT

A continuous process for producing ethyl acrylate and for recovering acrylic acid, ethyl acrylate, ethanol and water from an esterification reactor mixture containing acrylic acid, ethyl acrylate, ethanol, water, heavy ends, and acid catalyst; wherein a bottoms stream from a crude acrylic acid distillation column is fed to the reactor along with fresh acrylic acid.

### CROSS REFERENCE TO RELATED PATENT APPLICATIONS

This is a non-provisional application of prior pending U.S. provisional application Ser. No. 60/250,806 filed Nov. 17, 2003.

This invention relates to a method for combining acrylic acid (AA) and ethanol, and processing the reaction products to produce ethyl acrylate (EA) in improved yield.

A process for producing ethyl acrylate which has been practiced for more than one year by Rohm and Haas Company comprises the steps of:

a) feeding to the esterification reactor acrylic acid and ethanol, in a molar ratio of from 1 to 1.1 to 1 to 1.5, and the acid catalyst;

b) reacting the acrylic acid and ethanol to yield ethyl acrylate in a conversion of at least 90% on acrylic acid, and yielding the esterification reaction mixture comprising acrylic acid, ethyl acrylate, ethanol, ethyl acetate, water, heavy ends, and acid catalyst;

c) withdrawing a reactor bleed stream from the continuously converting esterification reactor mixture while concurrently sending a vaporized reactor mixture comprising acrylic acid, ethyl acrylate, ethanol and water from the esterification reactor to an ester distillation column;

d) feeding the reactor bleed stream, along with a crude acrylic acid bottoms stream, to a bleed stripper unit, from which a distillate comprising acrylic acid, ethyl acrylate and ethanol is removed and fed back to the reactor;

e) distilling from the ester distillation column, concurrently with above steps c) and d), a vaporized distillate mixture comprising ethyl acrylate, ethanol, and water;

f) condensing the vaporized distillate mixture to provide a first condensate comprising an organic phase and an aqueous phase; returning from 30 to 60 percent of the organic phase to the top of the ester distillation column;

g) feeding from 40 to 70 percent of the organic phase from step f) to an extraction unit along with caustic solution to produce an ethyl acrylate-rich organic phase low in acrylic acid and an aqueous phase;

h) feeding the ethyl acrylate-rich organic phase low in acrylic acid to a dehydration distillation column, which produces an overhead stream comprising ethyl acetate, ethanol and ethyl acrylate, and a bottom stream rich in ethyl acrylate;

i) condensing the overhead stream to provide a second condensate comprising an organic phase and an aqueous phase, and refluxing from 95 to 100% of the organic phase back to the dehydration distillation column; and

j) feeding combined aqueous streams from the first condensate, second condensate and the extraction unit to an alcohol stripper column, and recycling distillate from the alcohol stripper column to the esterification reactor.

The present invention relates to a method for running the ethyl acrylate process described above to obtain improved yield.

### Statement of the Invention

The present invention is directed to a continuous process for producing ethyl acrylate and for recovering acrylic acid, ethyl acrylate, ethanol and water from an esterification reactor mixture containing acrylic acid, ethyl acrylate, ethanol, water, heavy ends, and acid catalyst; said process comprising steps of:

a) feeding to the esterification reactor acrylic acid and ethanol, in a molar ratio of from 1 to 1.1 to 1 to 1.5, and the acid catalyst; wherein at least a portion of the acrylic acid is derived from a bottoms stream from a crude acrylic acid distillation column, said bottoms stream comprising from 60 to 90% acrylic acid;

b) reacting the acrylic acid and ethanol to yield ethyl acrylate in a conversion of at least 90% on acrylic acid, and yielding the esterification reaction mixture comprising acrylic acid, ethyl acrylate, ethanol, ethyl acetate, water, heavy ends, and acid catalyst;

c) withdrawing a reactor bleed stream from the continuously converting esterification reactor mixture while concurrently sending a vaporized reactor mixture comprising acrylic acid, ethyl acrylate, ethanol and water from the esterification reactor to an ester distillation column;

d) feeding the reactor bleed stream to a bleed stripper unit from which a distillate comprising acrylic acid, ethyl acrylate and ethanol is removed;

e) distilling from the ester distillation column, concurrently with above steps c) and d), a vaporized distillate mixture comprising ethyl acrylate, ethanol, and water;

f) condensing the vaporized distillate mixture to provide a first condensate comprising an organic phase and an aqueous phase; returning from 30 to 60 percent of the organic phase to the top of the ester distillation column;

g) feeding from 40 to 70 percent of the organic phase from step f) to an extraction unit along with caustic solution to produce an ethyl acrylate-rich organic phase low in acrylic acid and an aqueous phase;

h) feeding the ethyl acrylate-rich organic phase low in acrylic acid to a dehydration distillation column, which produces an overhead stream comprising ethyl acetate, ethanol and ethyl acrylate, and a bottom stream rich in ethyl acrylate;

i) condensing the overhead stream to provide a second condensate comprising an organic phase and an aqueous phase, and refluxing from 95 to 100% of the organic phase back to the dehydration distillation column; and

j) feeding combined aqueous streams from the first condensate, second condensate and the extraction unit to an alcohol stripper column, and recycling distillate from the alcohol stripper column to the esterification reactor.

### BRIEF DESCRIPTION OF THE DRAWING

FIG. 1 is a schematic depiction of equipment and flow lines used in a preferred embodiment of the invention.

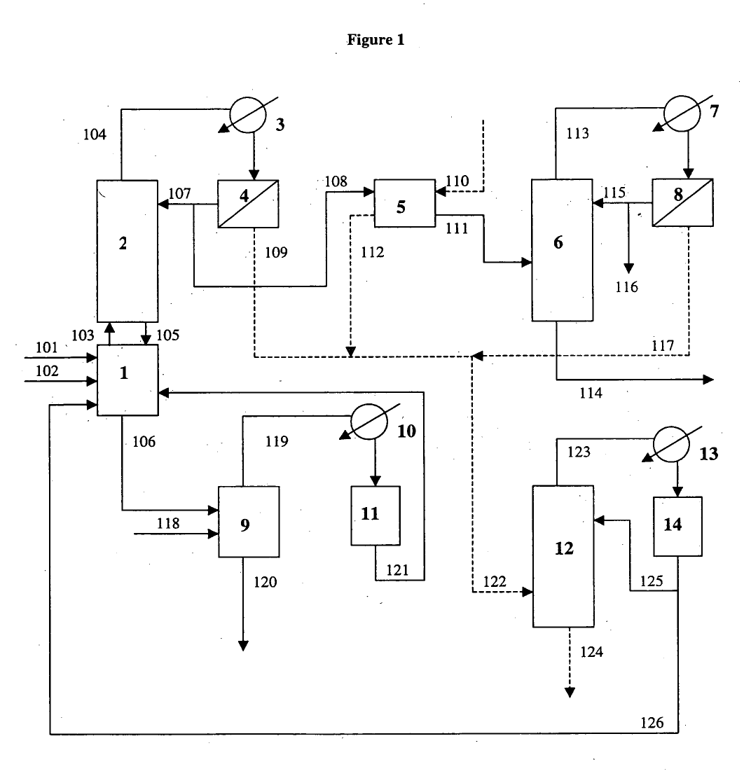

### DETAILED DESCRIPTION OF THE INVENTION

Fresh crude acrylic acid, ethanol, and esterification catalyst are fed to the esterification reactor 1 via line 101. A bottoms stream containing acrylic acid is also fed to the reactor via line 102 from a crude acrylic acid distillation column (not shown). Typical components of the bottoms stream comprise acrylic acid, at 60 to 90% and acrylic acid dimer (AOPA), at 10 to 40%. The acrylic acid from the bottoms stream comprises from 5% to 15% of the total acrylic acid fed to the esterification reactor. The molar ratio of acrylic acid to ethanol is from 1 to 1.1 to 1 to 1.5, preferably from 1 to 1.1 to 1 to 1.2. Esterification catalysts may be used at concentrations ranging from 4% to 8% by weight as measured in the esterification reactor bottoms. The esterification reactor temperature is maintained at from 85° C. to 105° C., at reactor pressures from 220-320 mm Hg. At least one heat exchanger may be used to control the temperature of esterification reactor 1. Desuperheated steam is preferred as the heat exchanger's heat source. In one embodiment, an external reboiler (not shown) is used as the heat exchanger for esterification reactor 1, and at least a portion of the reactor contents are passed through the reboiler via a reboiler recirculation line (not shown). Oxygen-containing gas, such as for example air, may be optionally admixed into the reboiler circulation line to ensure polymerization inhibitor efficacy within the esterification reactor 1 and its reboiler. Esterification reactor vaporized mixture is sent to ester distillation column 2 via line 103, where an azeotropic mixture of ethyl acrylate, ethanol and water, essentially free of acrylic acid, is distilled overhead through condenser 3 to phase separator 4 via line 104. Ester column bottoms, containing acrylic acid, is returned to reactor 1 via line 105, and a reactor bleed is sent to the bleed stripper 9 via line 106. A controlled portion of the separated organic layer is refluxed to the ester column 2 via line 107, while the remaining portion is sent forward to the extraction unit 5 via line 108. The aqueous layer from phase separator 4 is sent to the alcohol stripper 12 via line 109 for ethanol recovery.

The majority of the ethanol is extracted from ethyl acrylate and any residual acrylic acid is neutralized in extraction unit 5 using a very dilute caustic stream via line 110. The washed crude organic product, which contains ethyl acrylate, some water, and light-ends, is sent to dehydration distillation column 6 via line 111. The aqueous layer from extraction, which contains significant ethanol, is sent to the alcohol stripper 12 via line 112 for ethanol recovery.

Water and light-ends byproducts are distilled overhead in dehydration distillation column 6 to condenser 7 and phase separator 8 via line 113. Dehydration distillation column bottoms product comprising ethyl acrylate is sent forward for final processing to a product distillation column (not shown) via line 114. In one embodiment, such final processing is accomplished in one or more final product EA distillation columns (not shown) to provide final product ethyl acrylate meeting commercial quality specifications, and a bottoms stream comprising residual EA, inhibitors, and impurities. As with all polymerizable monomers, it is beneficial for the final product ethyl acrylate to comprise polymerization inhibitors in order to prevent polymerization in shipment and storage. MeHQ is most commonly utilized as a final product EA inhibitor, and is typically maintained at a concentration of between 10 ppm and 20 ppm in the final product EA. Inhibitor solutions comprising MeHQ may be provided to the final product distillation columns to achieve this final product inhibitor concentration. Oxygen-containing gas, such as for example air, may also be added to the final product distillation columns. In some embodiments, a variable amount of MeHQ inhibitor may be added directly to the ethyl acrylate product stream enroute to final product storage, thereby ensuring that the final product EA stream's inhibitor concentration remains within specification. The majority of the organic layer from phase separator 8 is refluxed back to the dehydration distillation column 6 via line 115, while up to 5% of the organic phase is periodically or continuously removed from the process to control light-ends build-up via line 116; preferably, the removed material is disposed of as waste. The aqueous layer from phase separator 8 is sent to the alcohol stripper 12 via line 117.

Reactor bleed is fed to the bleed stripper 9 via line 106, along with optional bottoms streams containing acrylic acid via line 118. In one embodiment of the invention, no acrylic acid-containing bottoms streams are fed to the bleed stripper. Bleed stripper overhead is sent to condenser 10 and receiver 11 via line 119, then it is recycled back to esterification reactor 1 via line 121. Bottoms residue from the bleed stripper is sent to an acid recovery unit (not shown) via line 120 for esterification catalyst recovery.

Mixed aqueous streams containing ethanol and some ethyl acrylate are sent to the alcohol stripper 12 via line 122. Organics are distilled overhead to condenser 13 and sent to receiver 14 via line 123. A controlled portion of the distillate is refluxed back to the alcohol stripper 12 via line 125, while the remaining distillate is recycled back to the esterification reactor 1 via line 126. The alcohol stripper bottoms wastewater is sent to a waste treatment plant (not shown) via line 124.

In order to prevent polymerization, an effective amount of one or more polymerization inhibitor may be added at any step in any component of the process. If additional inhibitor is required, any of a large number of known inhibitors may be used, for example, hydroquinone (HQ), 4-methoxyphenol (MEHQ), 4-ethoxyphenol, 4-propoxyphenol, 4-butoxyphenol, 4-heptoxyphenol, hydroquinone monobenzylether, 1,2-dihydroxybenzene, 2-methoxyphenol, 2,5-dichlorohydroquinone, 2,5-di-tert-butylhydroquinone, 2-acetylhydroquinone, hydroquinone monobenzoate, 1,4-dimercaptobenzene, 1,2-dimercaptobenzene, 2,3,5-trimethylhydroquinone, 4-aminophenol, 2-aminophenol, 2-N, N-dimethylaminophenol, 2-mercaptophenol, 4-mercaptophenol, catechol monobutylether, 4-ethylaminophenol, 2,3-dihydroxyacetophenone, pyrogallol-1,2-dimethylether, 2-methylthiophenol, t-butyl catechol, di-tert-butylnitroxide, di-tert-amylnitroxide, 2,2,6,6-tetramethyl-piperidinyloxy, 4-hydroxy-2,2,6,6-tetramethyl-piperidinyloxy, 4-oxo-2,2,6,6-tetramethyl-piperidinyloxy, 4-dimethylamino-2,2,6,6-tetramethyl-piperidinyloxy, 4-amino-2,2,6,6-tetramethyl-piperidinyloxy, 4-ethanoloxy-2,2,6,6-tetramethyl-piperidinyloxy, 2,2,5,5-tetramethyl-pyrrolidinyloxy, 3-amino-2,2,5,5-tetramethyl-pyrrolidinyloxy, 2,2,5,5-tetramethyl-1-oxa-3-azacyclopentyl-3-oxy, 2,2,5,5-tetramethyl-3-pyrrolinyl-1-oxy-3-carboxylic acid, 2,2,3,3,5,5,6,6-octamethyl-1,4-diazacyclohexyl-1,4-dioxy, salts of 4-nitrosophenolate, 2-nitrosophenol, 4-nitrosophenol, copper dimethyldithiocarbamate, copper diethyldithiocarbamate, copper dibutyldithiocarbamate, copper salicylate, methylene blue, iron, phenothiazine (PTZ), 3-oxophenothiazine, 5-oxophenothiazine, phenothiazine dimer, 1,4-benzenediamine, N-(1,4-dimethylpentyl)-N′-phenyl-1,4-benzenediamine, N-(1,3-dimethylbutyl)-N′-phenyl-1,4-benzenediamine, N-nitrosophenyl hydroxylamine and salts thereof, nitric oxide, nitrosobenzene, p-benzoquinone, copper naphthenate, copper acetate, manganese dimethyldithiocarbamate, manganese diethyldithiocarbamate, manganese dibutyldithiocarbamate, manganese naphthenate, manganese acetate, manganese acetylacetonate, cobalt acetate, cobalt carbonate, cobalt acetate, nitrogen dioxide, nitrobenzene, nitrosobutane, N-nitrosodiphenylamine, diphenylphenylenediamine, nitrosocarbazole, 1-nitroso-2-naphthol, 2,4 dinitrobenzene, triphenyl phosphine, triethyl phosphine, tributyl phosphine, triphenyl phosphite, triethyl phosphite, tri-iso-propylphosphite, tributyl phosphite, tricyclohexyl phosphite, sodium bisulfite, butyl mercaptan, dodecyl mercaptan, N,N-diethylhydroxylamine, or isomers thereof, mixtures of two or more thereof, mixtures of one or more of the above with molecular oxygen. The inhibitor(s) may be used alone or combined with a suitable diluent. The polymerization inhibitor is typically used at levels ranging from 100 ppm to 4,000 ppm by weight.

Because the alcohol stripper column may have sieve trays, a vapor phase inhibitor such as n-phenyl hydroxylamine or derivatives thereof may be useful. Liquid phase inhibitors may also be useful. In a preferred embodiment, the vapor phase inhibitor is added to the reboiler and the bottom trays of the column, while the liquid phase inhibitor is added to the top of the column. The amount of liquid phase inhibitor may range from 1 ppm to 1000 ppm, depending on the feed rate to the column.

In generating the crude EA, AA and ethanol are initially fed, along with acid catalyst, to an esterification reactor in a molar ratio of AA to ethanol in the range of 1:1.1 to 1:1.5, and reacted to a conversion on AA of at least 90%, using an acid catalyst of the mineral or sulfonic acid type, or a strong acid ion exchange resin; preferably sulfuric acid is used. The reactant ratio and EA conversion provide a crude EA stream which may be processed in the extraction unit and the dehydration distillation column. Reactor contents are maintained in a boiling state during continuous distillation of the vaporized mixture of AA, EA, ethanol and water.

The overall yield of ethyl acrylate was found to be at least 5% higher when the process was run as described above, as compared to the previous process in which acrylic acid distillation bottoms were fed to the bleed stripper instead of the reactor.

1. A continuous process for producing ethyl acrylate and for recovering acrylic acid, ethyl acrylate, ethanol and water from an esterification reactor mixture containing acrylic acid, ethyl acrylate, ethanol, water, heavy ends, and acid catalyst; said process comprising steps of:
a) feeding to the esterification reactor acrylic acid and ethanol, in a molar ratio of from 1 to 1.1 to 1 to 1.5, and the acid catalyst; wherein at least a portion of the acrylic acid is derived from a bottoms stream from a crude acrylic acid distillation column, said bottoms stream comprising from 60 to 90% acrylic acid;
b) reacting the acrylic acid and ethanol to yield ethyl acrylate in a conversion of at least 90% on acrylic acid, and yielding the esterification reaction mixture comprising acrylic acid, ethyl acrylate, ethanol, ethyl acetate, water, heavy ends, and acid catalyst;
c) withdrawing a reactor bleed stream from the continuously converting esterification reactor mixture while concurrently sending a vaporized reactor mixture comprising acrylic acid, ethyl acrylate, ethanol and water from the esterification reactor to an ester distillation column;
d) feeding the reactor bleed stream to a bleed stripper unit from which a distillate comprising acrylic acid, ethyl acrylate and ethanol is removed;
e) distilling from the ester distillation column, concurrently with above steps c) and d), a vaporized distillate mixture comprising ethyl acrylate, ethanol, and water;
f) condensing the vaporized distillate mixture to provide a first condensate comprising an organic phase and an aqueous phase; returning from 30 to 60 percent of the organic phase to the top of the ester distillation column;
g) feeding from 40 to 70 percent of the organic phase from step f) to an extraction unit along with caustic solution to produce an ethyl acrylate-rich organic phase low in acrylic acid and an aqueous phase;
h) feeding the ethyl acrylate-rich organic phase low in acrylic acid to a dehydration distillation column, which produces an overhead stream comprising ethyl acetate, ethanol and ethyl acrylate, and a bottom stream rich in ethyl acrylate;
i) condensing the overhead stream to provide a second condensate comprising an organic phase and an aqueous phase, and refluxing from 95 to 100% of the organic phase back to the dehydration distillation column; and
j) feeding combined aqueous streams from the first condensate, second condensate and the extraction unit to an alcohol stripper column, and recycling distillate from the alcohol stripper column to the esterification reactor.
